(19) United States
(12) Plant Patent Application Publication
(10) Pub. No.: US 2003/0070199 P1
(43) Pub. Date: Apr. 10, 2003

(54) OSTEOSPERMUM PLANT NAMED 'KAKEGAWA AU8'

(76) Inventor: Masao Kanno, Chigasaki City (JP)

Correspondence Address: JONDLE & ASSOCIATES P.C., 9085 EAST MINERAL CIRCLE, SUITE 200, CENTENNIAL, CO, 80112, US

(21) Appl. No.: 09/971,676

(22) Filed: Oct. 9, 2001

(51) Int. Cl. A01H 5/00

(52) U.S. Cl. PLT/360

(57) ABSTRACT

An Osteospermum cultivar particularly distinguished by its violet flower color and tendency to hold flowers open later in the evening.

### BACKGROUND OF THE NEW PLANT

The present invention originated in 1994 from an F₁ selection of Osteospermum fruiticosum. The female parent was known as breeding line No. 375 and possessed large, white flowers. The male parent was known as line No. 471 and possessed large flowers that would stay open later into the evening than most Osteospermums. Line No. 375 originated from a hybridization made in 1991 between an F₅ selection from a cross between a pink flowered line and a white with blue eye line, and a dwarf habit breeding line with flowers that stayed open late. Line No. 471 originated from a hybridization made in 1993 between a dwarf, rose flowered line with flowers that stayed open late and a dwarf, pink flowered line. The origin of these lines dates back to crosses made by the inventor in 1984. The original selection made in 1995 was vegetatively propagated again in 1996 and stability was confirmed in 1997. The line was established as ‘Kakegawa AU8’, and determined to have its characteristics firmly fixed.

### DESCRIPTION OF PHOTOGRAPH

This new Osteospermum plant is illustrated by the accompanying photograph which shows blooms, and foliage of the plant in full color, the colors shown being as true as can be reasonably obtained by conventional photographic procedures.

FIG. 1 shows a view of three plants propagated in a 20 cm diameter pot;

FIG. 2 shows a close-up view of a single inflorescence.

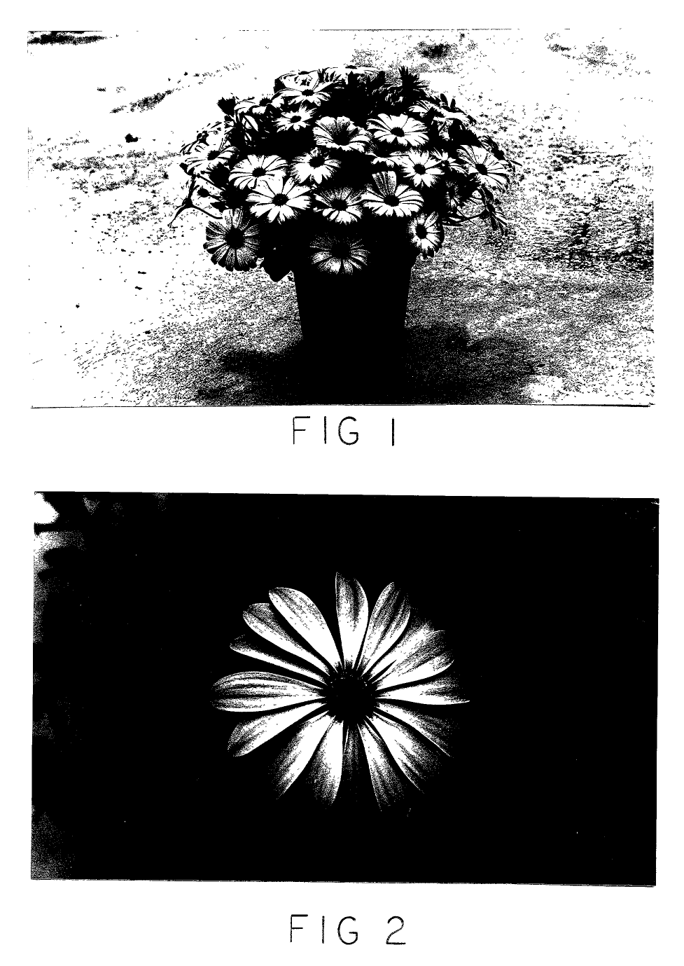

### DESCRIPTION OF THE NEW CULTIVAR

The following detailed descriptions set forth the distinctive characteristics of ‘Kakegawa AU8’. The data which defines these characteristics were collected from asexual reproductions carried out in Salinas, California. Three plants from fully rooted 20 cm diameter pots and grown in the same conditions. Data was collected on plants 28 weeks from transplanting rooted cuttings into 16 cm diameter pots and growing as described below. Color references are to the RHS Colour Chart of The Royal Horticultural Society of London (RHS).

### DESCRIPTION OF THE NEW PLANT

Classification:

Family: Compositae

Botanical: Osteospermum fruticosum

Commercial: Cape Daisy

Parentage:

Female parent—breeding line No. 375 (unpatented)

Male parent - breeding line No. 471 (unpatented)

Growth:

Time to produce a rooted cutting - Cuttings will colonize a 2.5 cm diameter by 2.5 cm tall greenhouse tray cell with peat-based plant media in approximately five weeks. Cuttings are dipped in a normal dilution (1:9) of Dip ‘N Grow™ root inducing solution in water. The trays are misted hourly during rooting.

Environmental conditions for plant growth—Rooted cuttings are transplanted to pots with a 16 cm diameter, one plant per pot. Peat moss-based growing media is used. The pots are watered using a 150-200 ppm fertilizer solution containing 18% nitrogen, 8% phosphorus and 18% potassium. The soil is allowed to dry between waterings. During the first few weeks after transplanting the plants should have evening temperatures around 15-18° C. for good root growth. When plants reach 7.5-10 cm in height they are pinched back to 5-6 leaves to promote branching. The plants are grown through the winter in cool greenhouses (10-15° C.) with no shading to keep their habit compact. In spring, after the plants have received at least four weeks of cool conditions to vernalize, warmer temperatures can be used to promote blooming. Spring and summer daytime high temperatures in Salinas, California, where the data was collected, range from 16-25° C.

Time to bloom from propagation—18-20 weeks when rooted vegetative cuttings are transferred to a 16 cm diameter pot in late Fall and given several weeks of below 50° F. temperature prior to increasingly warmer spring weather.

Habit.—vigorous, well branched

Life cycle.—perennial

Plant:

Form.—upright

Plant Size.—height is 35-40 cm; width is 35-40 cm

Stems:

Internode length.—0.8-1.0 cm

Color.—Yellow-green (RHS N 144A)

Description.—Strong, erect, herbaceous

Stem diameter.—3.0-4.0 mm

Pubescence.—Short, transparent

Leaves:

Arrangement.—alternate

Shape.—spatulate

Apex.—mucronate

Base.—oblique, sessile

Margin.—dentate

Venation.—pinnate

Color.—Upper is green (RHS 137A); Lower is green (RHS 137C)

Inflorescence:

Type.—Capitulum (head); disk flowers are staminate and ray flowers are carpellate.

Diameter.—7.0-8.0 cm

Habit.—Determinate

Fragrance.—None

Fruit and seed.—None

Calyx.

Description.—13-16 inflorescence bracts, arranged symmetrically

Shape.—Linear

Apex.—Acute

Size.—Length is 1.2-1.4 cm; Width is 2.0-3.0 mm

Margin.—Entire

Color.—Upper is green (RHS 137D) and lower is green (RHS 138C)

Ray florets.

Corolla.—One petal per flower on outer whorl of flowers

Petals.—14-18

Petal size.—Length of petals is 3.8-4.0 cm; width is 1.0 cm

Petal shape.—Spatulate

Petal apex.—Obtuse with indentation at tip

Petal margin.—Entire

Petal color.—Ventral surface of ray petals is red-purple (RHS 70B) with white shading; dorsal surface of petal is purple (RHS 79 C) with purple-violet (RHS N82C) veins.

Ovary.—Inferior

Style form.—One style with two stigma branches

Style color.—Purple (RHS 75 B)

Stigma color.—Purple (RHS N79 A)

Disk florets:

Bud shape.—Tubular

Bud size.—Length is 5 mm and width is 1 mm

Color of bud tip.—Violet-blue (RHS N93 A)

Number per head.—30-35

Anther color.—Purple (RHS N77 A)

Filament color.—Yellow (RHS 4 D)

Pollen color.—Yellow-orange (RHS 17 A)

Disease and Insect Resistance

No susceptibility to diseases or insects noted to date.

Comparison with Known Cultivars

‘Kakegawa AU8’ is most similar to the variety ‘Kakegawa AU2’, the subject of U. S. Plant patent application Ser. No. 09/657,540 which is marketed under the name ‘Sea Mist Pink and White’. The two plants differ as shown in the table below.

| Characteristic | ‘Kakegawa AU8’ | ‘Kakegawa AU2’ |
|---|---|---|
| Leaf margin | Highly serrated | Slightly serrated |
| Flower | 7.0-8.0 cm | 5.5-6.0 cm |
| diameter | | |
| Petal length | 3.8-4.0 cm | 2.5-3.0 cm |
| Petal color | Ventral surface is | Ventral surface is red-purple (RHS |
| | red-purple (RHS | 70B) with the lower portion of the |
| | 70B) with white | petal white (RHS 1550); dorsal |
| | shading; dorsal | surface is red-purple (RHS 71A) |
| | surface of | |
| | petal purple | |
| | (RHS 79C) | |
| Veins | On dorsal petal | On dorsal petal surface are violet |
| | surface are | (RHS 83B) |
| | purple-violet | |
| | (RHS N82C) | |

I claim:

1. A new and distinct cultivar of Osteospermum as shown and described herein.
